(19) United States
(12) Patent Application Publication
(10) Pub. No.: US 2026/0088033 A1
(43) Pub. Date: Mar. 26, 2026

(54) METHOD FOR CONVERTING ANIMAL LANGUAGE, ELECTRONIC DEVICE AND STORAGE MEDIUM

(71) Applicant: Beijing Baidu Netcom Science Technology Co., Ltd., Beijing (CN)

(72) Inventors: Chengcheng Wu, Beijing (CN); Ye Zhang, Beijing (CN); Baoju Liu, Beijing (CN); Zeyu Wang, Beijing (CN)

(21) Appl. No.: 19/407,666

(22) Filed: Dec. 3, 2025

(30) Foreign Application Priority Data
Dec. 6, 2024 (CN) CN202411793938.3

(51) Int. Cl. G10L 17/26; G10L 17/18; G10L 25/63

(52) U.S. Cl. CPC G10L 17/26; G10L 17/18; G10L 25/63

(57) ABSTRACT

Provided is a method for converting animal language, an electronic device and a storage medium, relating to the field of artificial intelligence technology, and specifically to the fields of machine learning, deep learning, natural language processing and other technologies. The method includes: obtaining multimodal data related to an animal, wherein the multimodal data comprises animal sound data, animal behavior data and animal physical sign data; preprocessing the multimodal data to obtain fused multimodal data; recognizing current emotion of the animal according to the fused multimodal data to obtain an emotion recognition result of the animal; and performing semantic mapping and language translation on the emotion recognition result to convert animal language into human language to obtain a language conversion result.

The present application claims priority to Chinese Patent Application No. CN202411793938.3, filed with the China National Intellectual Property Administration on December 6, 2024, the disclosure of which is hereby incorporated herein by reference in its entirety.

### TECHNICAL FIELD

The present disclosure relates to the field of artificial intelligence technology, specifically to the fields of machine learning, deep learning, natural language processing and other technologies, and particularly to a method and an apparatus for converting animal language, an electronic device and a storage medium.

### BACKGROUND

Current technologies on the market attempt to interpret the emotional world of animals through some basic devices for translating animal sounds and behaviors as well as pet emotion analysis tools that utilize the artificial intelligence image recognition technology. However, these methods are often limited to superficial interpretation of animal behaviors. These technologies cannot delve into the complex emotional level of animals, and cannot achieve deep, real-time emotional understanding and interactive communication between humans and animals.

The present disclosure provides a method and an apparatus for converting animal language, an electronic device and a storage medium.

According to one aspect of the present disclosure, provided is a method for for converting animal language, including: obtaining multimodal data related to an animal, where the multimodal data includes animal sound data, animal behavior data and animal physical sign data; preprocessing the multimodal data to obtain fused multimodal data; recognizing current emotion of the animal according to the fused multimodal data to obtain an emotion recognition result of the animal; and performing semantic mapping and language translation on the emotion recognition result to convert animal language into human language to obtain a language conversion result.

According to another aspect of the present disclosure, provided is an apparatus for converting animal language, including: an obtaining module configured to obtain multimodal data related to an animal, where the multimodal data includes animal sound data, animal behavior data and animal physical sign data; a preprocessing module configured to preprocess the multimodal data to obtain fused multimodal data; an emotion recognition module configured to recognize current emotion of the animal according to the fused multimodal data to obtain an emotion recognition result of the animal; and a conversion module configured to perform semantic mapping and language translation on the emotion recognition result to convert animal language into human language to obtain a language conversion result.

According to a third aspect of the present disclosure, provided is an electronic device, including: at least one processor; and a memory connected in communication with the at least one processor; where the memory stores an instruction executable by the at least one processor, and the instruction, when executed by the at least one processor, enables the at least one processor to execute the method described in any one of the above-mentioned technical solutions.

According to a fourth aspect of the present disclosure, provided is a non-transitory computer-readable storage medium storing a computer instruction thereon, and the computer instruction is used to cause a computer to execute the method described in any one of the above-mentioned technical solutions.

According to a fifth aspect of the present disclosure, provided is a computer program product including a computer program, and the computer program implements the method described in any one of the above-mentioned technical solutions, when executed by a processor.

The present disclosure provides the method and apparatus for converting animal language, the electronic device and the storage medium. The present disclosure can achieve the comprehensive capture and accurate recognition of animal emotions by obtaining the multimodal data such as animal sounds, behaviors and physical signs. Next, the emotional states and intentions of animals are converted into language that humans can understand through semantic mapping and language translation technologies, thereby greatly enhancing the communication ability between humans and animals, improving the accuracy in understanding animal emotions and the real-time nature of interaction, and providing humans with a brand-new way to communicate with animals. That is, this solution can accurately recognize current emotional states of animals and convert them into human language, thereby achieving deeper emotional communication and understanding between animals and humans, and improving the accuracy and efficiency of cross-species communication.

It should be understood that the content described in this part is not intended to identify critical or essential features of embodiments of the present disclosure, nor is it used to limit the scope of the present disclosure. Other features of the present disclosure will be easily understood through the following description.

### BRIEF DESCRIPTION OF THE DRAWINGS

The accompanying drawings are used to better understand the present solution, and do not constitute a limitation to the present disclosure.

FIG. 1 is a schematic diagram of steps of a method for converting animal language in an embodiment of the present disclosure;

FIG. 2 is a schematic diagram of a process of collecting the multimodal data in an embodiment of the present disclosure;

FIG. 3 is a schematic diagram of a process of preprocessing the multimodal data in an embodiment of the present disclosure;

FIG. 4 is a schematic diagram of a process of obtaining the emotion recognition result of the animal in an embodiment of the present disclosure;

FIG. 5 is a schematic diagram of a process of converting animal language into human language in an embodiment of the present disclosure;

FIG. 6 is a schematic diagram of a process of updating the emotion tag in an embodiment of the present disclosure;

FIG. 7 is a principle block diagram of an apparatus for converting animal language in an embodiment of the present disclosure; and

FIG. 8 is a block diagram of an electronic device for implementing the method for converting animal language in an embodiment of the present disclosure.

### DETAILED DESCRIPTION

Hereinafter, descriptions to exemplary embodiments of the present disclosure are made with reference to the accompanying drawings, include various details of the embodiments of the present disclosure to facilitate understanding, and should be considered as merely exemplary. Therefore, those having ordinary skill in the art should realize, various changes and modifications may be made to the embodiments described herein, without departing from the scope and spirit of the present disclosure. Likewise, for clarity and conciseness, descriptions of well-known functions and structures are omitted in the following descriptions.

At present, the technologies related to communication between humans and animals on the market mainly divided into two categories as follows:

The first approach is to use a simple translation device for animal sounds and behaviors, which realizes the function of translating animal emotions mainly based on "voiceprint database + simple algorithm". Typical examples are some pet behavior and sound recognition devices on the market, which can use relatively simple sensors to capture sounds and some typical movements of animals, and then match them to a pre-built emotion database for simple emotion mapping, such as recognizing that a dog's bark represents a request for food, anger, etc.

The second approach is a pet emotion analysis tool based on AI (Artificial Intelligence) image recognition, that is, introducing the image processing and AI technologies to help users understand the pets' emotional responses. For example, some companies capture animal faces and movements in the camera image data, and then combine existing deep learning-trained models to analyze different expressions of animal faces and identify specific expressions such as laughter, anger, grievance and confusion.

Both of the above-mentioned approaches have several limitations in understanding and translating animal emotions: firstly, the emotion translation is simplistic, relies on the pre-set voiceprint data and behavior classification, and cannot continuously track emotional changes, resulting in insufficient accuracy in complex scenarios; secondly, the lack of multimodal fusion analysis and over-reliance on a single information source limit the comprehensiveness and accuracy of emotion translation; furthermore, insufficient temporal detection and the lack of ability to continuously track the emotional state lead to dulled perception of emotional changes; moreover, the lack of adaptive learning and optimization mechanisms makes it difficult to optimize and iterate when facing unknown emotional patterns, limiting the system's flexible scalability; and finally, the inability to perform edge computing results in insufficient real-time performance, and affects instant interaction between humans and animals. These limitations collectively lead to the inadequacy of the existing technologies in achieving deep and real-time cross-species emotional communication.

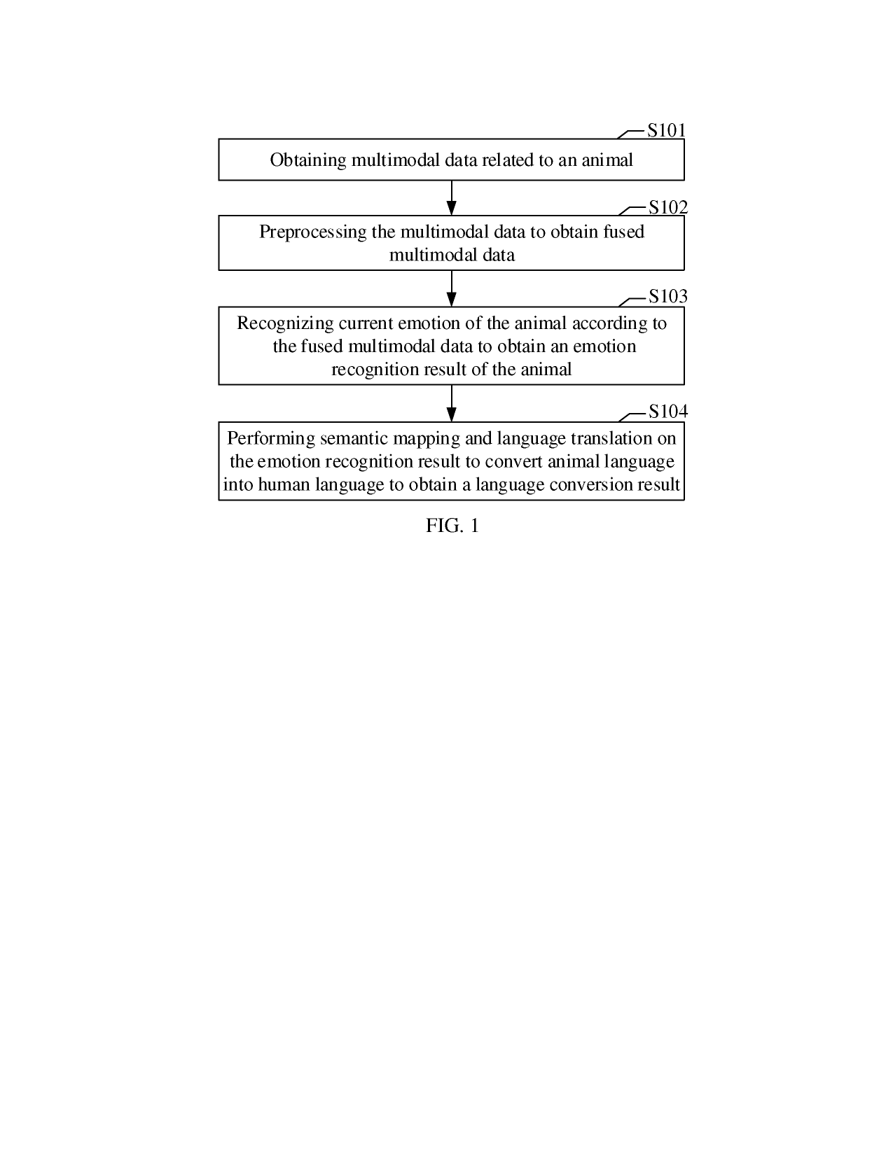

In order to address the above technical problems, the present disclosure provides a method for converting animal language. Referring to FIG. 1, which is a schematic diagram of steps of the method for converting animal language in an embodiment of the present disclosure, this method can be applied to a server side and includes:

Step S101: obtaining multimodal data related to an animal, where the multimodal data includes animal sound data, animal behavior data and animal physical sign data.

Specifically, obtaining the multimodal data related to the animal refers to collecting different types of information through multiple sensors to gain a comprehensive understanding of the animal's state and emotion. Here, the "animal sound data" refers to various sounds made by the animal captured by an audio sensor, and these sounds can reflect the animal's emotion and requirement; the "animal behavior data" refers to body movements and postures of the animal recorded by a video camera, and helps to analyze the animal's behavioral pattern and emotional expression; and the "animal physical sign data" refers to physiological indicators such as heart rate and body temperature of the animal monitored by a physiological sensor, and these indicators can provide physiological evidence of the animal's emotional state.

In this way, the animal emotions and behaviors can be comprehensively captured by obtaining the animals' multimodal data including sound, behavior and physical sign data, thereby providing more accurate emotion analysis and behavior understanding, enhancing communication and interaction between humans and animals, and improving animal welfare and human response capability to animal behaviors.

Step S102: preprocessing the multimodal data to obtain fused multimodal data.

Specifically, preprocessing the multimodal data refers to performing a series of processing steps on the raw data collected from different sources (such as audio, video and physiological sensors), including, for example, performing noise reduction, normalization and feature extraction on the multimodal data to facilitate analysis and understanding. The "fused multimodal data" refers to integrating the preprocessed data into a unified dataset. This process involves time alignment (ensuring synchronization of all data over time), feature fusion (merging features in different modalities into a comprehensive feature vector), and data synchronization (handling differences in timestamp and sampling rate between different data streams). This preprocessing and fusion process can provides a comprehensive data view, making subsequent emotion recognition and language translation more accurate and efficient.

In this way, by preprocessing the multimodal data such as animal sounds, behaviors and physical signs and then integrating these data into a unified dataset, the consistency and usability of the data can be improved, thereby making the emotion recognition and language translation more accurate, enhancing the system's comprehensive understanding of animal behaviors and emotional states, and improving the efficiency and effectiveness of cross-species communication.

Step S103: recognizing current emotion of the animal according to the fused multimodal data to obtain an emotion recognition result of the animal.

Specifically, after the fused multimodal data is obtained, the current emotion of the animal is recognized based on the fused multimodal data. Specifically, the comprehensive dataset integrating information such as animal sounds, behaviors and physical signs is used to analyze and judge the animal's emotional state by the machine learning and deep learning technologies. The "emotion recognition" here refers to recognizing the emotional state of the animal, such as anxiety, excitement or relaxation, by analyzing the features extracted from these multimodal data. To obtain the emotion recognition result of the animal, the fused data may be for example input into a trained emotion recognition model. This model outputs the emotion recognition result of the animal by comparing the features of known emotion states, thus providing a basis for subsequent language conversion and human-computer interaction.

In this way, it is conducive to accurately recognizing the animal's current emotional state by analyzing the fused multimodal data, including the animal's sounds, behaviors and physical signs. This process not only improves the accuracy in understanding and responding to animal emotions, but also enhances communication between humans and animals, enabling humans to better interpret animal needs and emotions, and thereby improving the animal welfare and the closeness of the relationship between humans and pets.

Step S104: performing semantic mapping and language translation on the emotion recognition result to convert animal language into human language to obtain a language conversion result.

Specifically, "performing semantic mapping and language translation on the emotion recognition result" refers to the process of converting the animal emotional state such as anxiety, excitement or happiness obtained by analyzing the multimodal data into the language expression that humans can understand. Specifically, this process involves using a pre-trained language model and deep learning techniques to establish a correspondence between emotional features of animals and corresponding expressions in human language, i.e., "semantic mapping". Subsequently, these mapping results are converted into specific text or speech output, to achieve "language translation". Ultimately, the "language conversion result" refers to conversion of the animal's emotion and intention into the form of human language, enabling the human user to intuitively understand the animal's "language" and thus achieve effective cross-species communication.

In this way, the nonverbal communication of the animal can be converted into language that humans can understand by performing semantic mapping and language translation on the emotion recognition result. This process not only breaks down communication barriers between humans and animals, but also greatly enhances human understanding of animal emotions and needs, making interactions between humans and animals more harmonious, and also providing new perspectives and tools for animal welfare and behavioral research.

The present disclosure provides the method and apparatus for converting animal language, the electronic device and the storage medium. The present disclosure can achieve the comprehensive capture and accurate recognition of animal emotions by obtaining the multimodal data such as animal sounds, behaviors and physical signs. Next, the emotional states and intentions of animals are converted into language that humans can understand through semantic mapping and language translation technologies, thereby greatly enhancing the communication ability between humans and animals, improving the accuracy in understanding animal emotions and the real-time nature of interaction, and providing humans with a brand-new way to communicate with animals. That is, this solution can accurately recognize current emotional states of animals and convert them into human language, thereby achieving deeper emotional communication and understanding between animals and humans, and improving the accuracy and efficiency of cross-species communication.

In some optional embodiments, the step of obtaining the multimodal data related to the animal includes: collecting sound wave information emitted by the animal to obtain the animal sound data; collecting body language and movement change of the animal to obtain the animal behavior data; and collecting physical and biological indicators of the animal to obtain the animal physical sign data.

Specifically, the sounds made by the animal are captured by an audio collector to collect the sound wave information emitted by the animal, to obtain the "animal sound data"; and simultaneously, the body language and movement changes of the animal are collected by a visual sensor such as a video camera to form the "animal behavior data", where these data reflect the activities and nonverbal behaviors of the animal; and moreover, the "animal physical sign data" such as heart rate and body temperature of the animal are monitored by a physiological sensor, where these physical and biological indicators provide important information for understanding the physiological state and emotion of the animal. By integrating these multimodal data, the communication pattern and emotional state of the animal can be comprehensively captured, laying the foundation for further data analysis and emotion recognition.

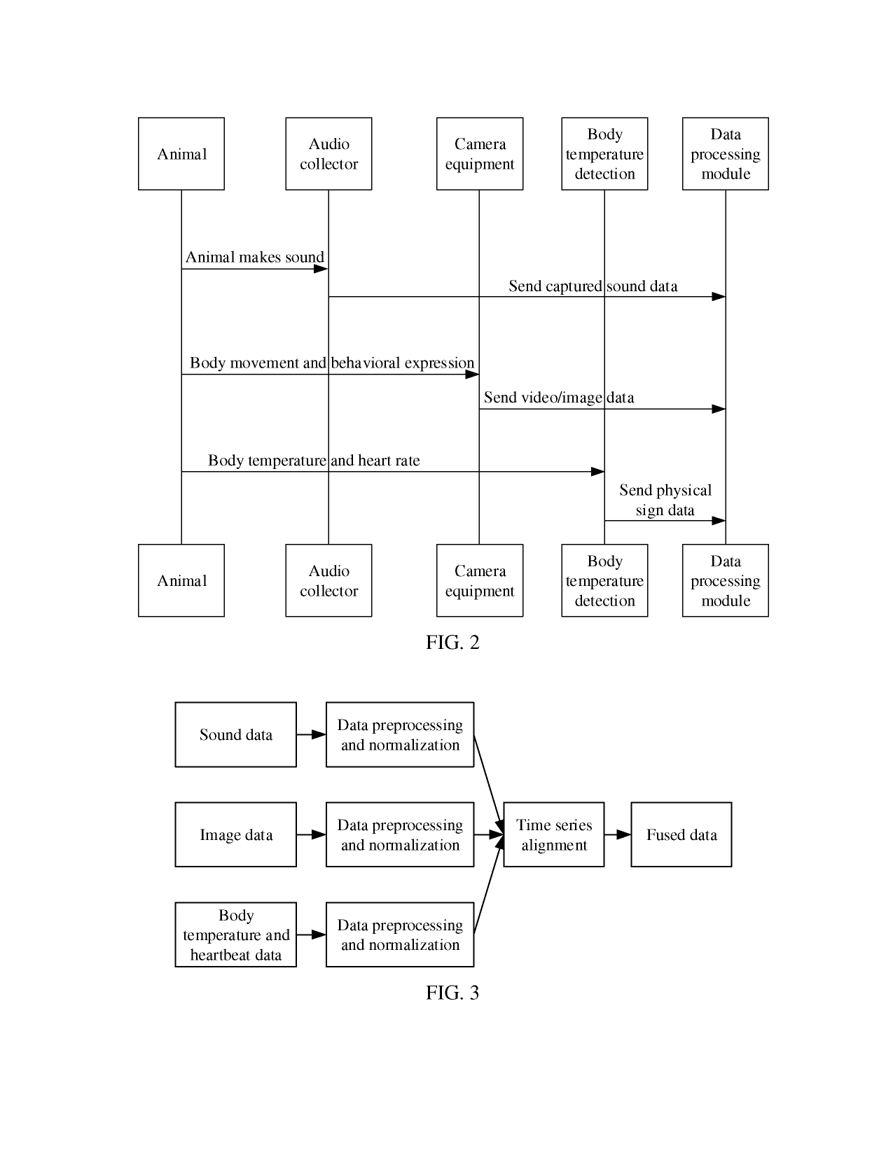

To facilitate understanding of the solution in the embodiments of the present disclosure, an example is given below, as shown in FIG. 2. FIG. 2 is a schematic diagram of a process of collecting the multimodal data in an embodiment of the present disclosure. First, an audio collector is used to capture the sounds make by the animal in real time, and then the audio collector sends the captured sound data to a data processing module. The data processing module can use a corresponding audio filter to process noise and reduce background interference. For example, for dog barks, the pitch, amplitude, duration, frequency, breakpoint change and other dimension information are sampled together to capture all the information of the barks.

For collection of the animal behavior data, the camera equipment (such as a high-definition camera and an infrared camera) obtains the body movements and behavioral expressions (such as tail wagging, jumping, lying down and other posture analysis) of the animal, supplemented with specific animal body characteristics (upright ears, dilated pupils, etc.). Then the captured video/image data is sent to the data processing module.

For collection of the animal physical sign data, a high-precision contact or non-contact body temperature detection sensor collects the heart rate and body temperature, and sends the collected physical sign data to the data processing module.

In this way, the relatively comprehensive sound data, behavior data and physical sign data of the animal are obtained by collecting the sound wave information, body language and movement change emitted by the animal as well as the physical and biological indicators. The integration of the multi-dimensional information provides a rich and accurate data foundation for in-depth understanding and analysis of the animal's emotion and behavior, thereby enabling humans to more accurately interpret the animal's communication intention and physiological state, strengthening nonverbal communication between humans and animals, improving the effectiveness of animal care and training, and also opening up new avenues for animal health monitoring and behavioral research.

In some optional embodiments, the step of preprocessing the multimodal data to obtain the fused multimodal data includes: denoising the multimodal data for data cleaning to obtain cleaned multimodal data; normalizing the cleaned multimodal data to obtain normalized multimodal data; and performing time series alignment and fusion on the normalized multimodal data to obtain the fused multimodal data.

Specifically, "denoising the multimodal data for data cleaning" refers to using signal processing techniques to remove noise and interference from the audio and video data, to improve the data quality and obtain the cleaned multimodal data. Next, "normalizing the cleaned multimodal data" means converting data from different sources and at different scales into a uniform format or scale so that the machine learning model can process the data more effectively. This step results in the normalized multimodal data. Finally, "performing time series alignment and fusion on the normalized multimodal data" involves aligning data in different modalities in time and merging them into a unified dataset. This step ensures the temporal consistency of the data and integrates information from different sensors, ultimately resulting in the fused multimodal data to provide an accurate and comprehensive data foundation for subsequent emotion recognition and behavior analysis.

To facilitate understanding of the solution in the embodiments of the present disclosure, an example is given below, as shown in FIG. 3. FIG. 3 is a schematic diagram of a process of preprocessing the multimodal data in an embodiment of the present disclosure. First, the sound data, image data, and body temperature and heart rate data in the multimodal data are preprocessed and normalized respectively. The processing includes noise reduction and data cleaning, used to handle invalid parts in the audio and visual information, for example, filter out noise (wind, voice, etc.) that may be generated by humans to make the audio signal clearer and easier to recognize. In terms of images, background movements and objects in video frames are removed to retain only key information, such as behavior changes of the animal. Next, the data is normalized. Whether the data is an audio signal, a video or physical sign information, it needs to be normalized, transformed into a unified standard and expressed as a feature vector that can be processed by machine learning algorithms.

Finally, the time series alignment and fusion are performed on the multimodal data. The data alignment is a prerequisite for data fusion, and requires solving the problem of temporal and spatial differences between signals in different modalities when collected. For example, a dog barking event often has a corresponding time difference with the moment of accompanying a body movement or a body temperature fluctuation. These data must be time-calibrated before input, so that the multimodal inputs in the translation task have a consistent reference point.

In this way, the quality, consistency and usability of the data can be significantly improved by performing denoising, normalization, time series alignment and fusion on the multimodal data, thereby making the features extracted from animal sounds, behaviors and physical signs more accurate and reliable. This process not only enhances the accuracy of data analysis but also improves the model's precision in recognizing animal emotions and behaviors, providing a solid data foundation for achieving efficient and accurate cross-species communication.

In some optional embodiments, the step of recognizing the current emotion of the animal according to the fused multimodal data to obtain the emotion recognition result of the animal includes: performing sound feature extraction, visual motion feature extraction and physical sign change analysis on the fused multimodal data by a deep learning model to obtain a multimodal feature vector; and performing emotion analysis on the multimodal feature vector by a generative adversarial network to obtain the emotion recognition result of the animal.

Specifically, "performing sound feature extraction, visual motion feature extraction and physical sign change analysis on the fused multimodal data by a deep learning model" refers to using advanced deep learning techniques, such as Convolutional Neural Network (CNN) and Recurrent Neural Network (RNN), to process and analyze the multimodal dataset integrating the sound, visual and physiological data. This process involves extracting sound features, such as pitch, rhythm and volume, from audio signals; extracting visual motion features, such as posture and behavioral pattern, from the video data; and analyzing changes in physical signs, such as heart rate and body temperature fluctuation, from the physiological data. These features together constitute a multimodal feature vector, which comprehensively reflects the animal's physiological and behavioral state. Next, "performing emotion analysis on the multimodal feature vector by a generative adversarial network" refers to using a deep learning framework such as Generative Adversarial Network (GAN) to enable the network to recognize and distinguish different emotional states through the adversarial training process. This process ultimately produces the emotion recognition result of the animal, converts the complex emotion and behavior of the animal into an understandable emotion tag, and provides a basis for further semantic mapping and language translation.

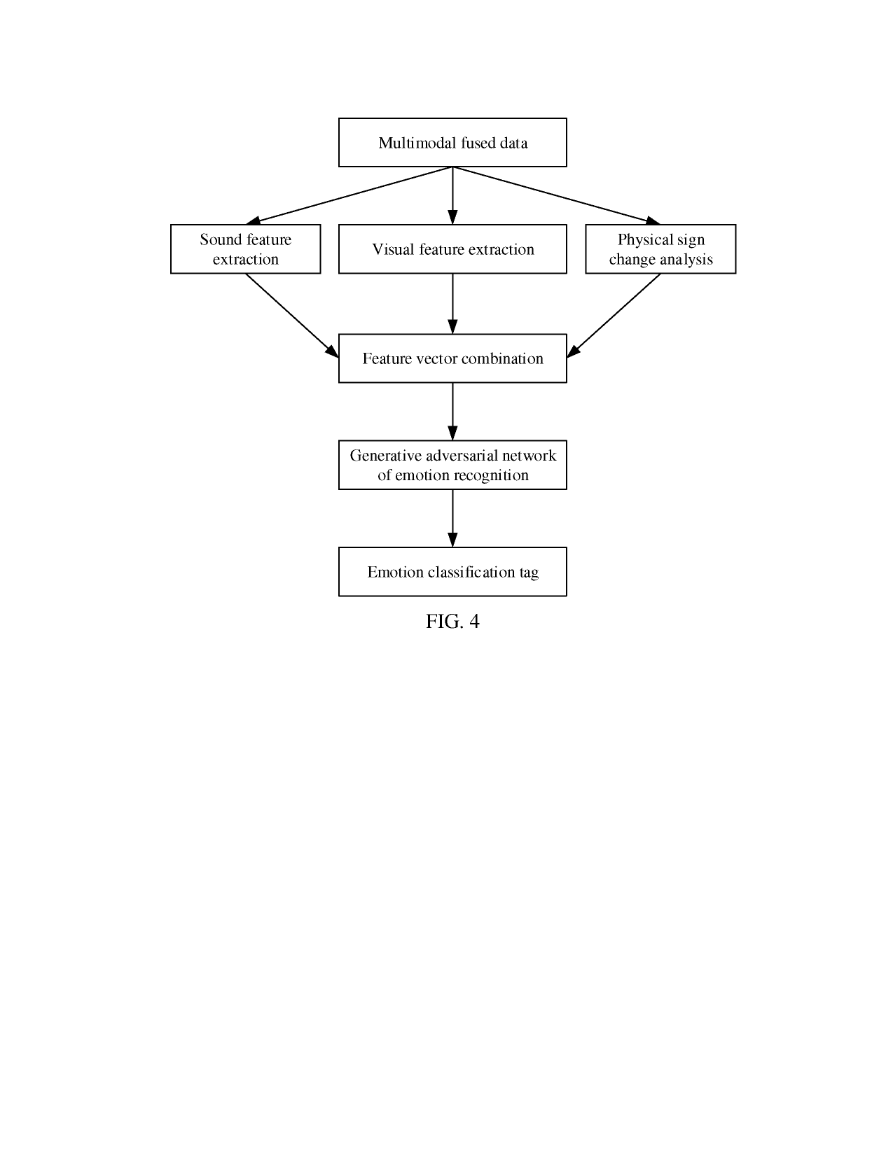

To facilitate understanding of the solution in the embodiments of the present disclosure, an example is given below, as shown in FIG. 4. FIG. 4 is a schematic diagram of a process of obtaining the emotion recognition result of the animal in an embodiment of the present disclosure. After the fused multimodal data is obtained, fine-grained feature extraction is performed on the data of each modality using the deep learning model. Specifically, the deep learning model extracts sound features and visual motion features and analyzes physical sign changes to obtain a combination of feature vectors. Next, a large model based on Generative Adversarial Network (GAN) of emotion recognition is used to analyze the voiceprint features, motion changes and physical sign fluctuations in the data to obtain an emotion classification tag. For example, when detecting a low-frequency barking of an animal accompanied by tense limbs and dilated pupils, the model identifies the animal as being in a state of high alert through comparative reasoning, and infers the possible underlying psychological activity (fear or confusion) through further feature association.

In this way, the use of the deep learning model for comprehensive analysis of the fused multimodal data can accurately extract features such as sounds, visual movements and physical sign changes of the animal to forming the multimodal feature vector. Then, the generative adversarial network is used to conduct in-depth emotion analysis of these features, thereby obtaining the emotion recognition result of the animal. This process not only improves the accuracy and depth of emotion recognition, but also enhances the understanding of animal behavior and psychological state, providing strong technical support for more effective human-animal communication.

In some optional embodiments, the step of performing semantic mapping and language translation on the emotion recognition result to convert animal language into human language to obtain the language conversion result includes: extracting an emotion tag and a sound feature from the emotion recognition result, and converting the sound feature into a standardized sound vector; mapping the emotion tag and the sound vector semantically by a pre-trained language model to obtain an emotion intention; and performing language translation on the emotion intention by a language generator to generate corresponding human language, to obtain the language conversion result.

Specifically, the "pre-trained language model" refers to an artificial intelligence model that has been trained with a large amount of data and is capable of understanding and processing natural language. This model is used here to perform "semantic mapping" on the animal's emotion tag (i.e., the animal emotional state obtained from the emotion recognition module) and sound vector, that is, correspond the animal's sound feature to the emotional semantics in human language, so as to recognize the animal's emotion intention. Next, the "language generator" is the process of converting the animal's nonverbal communication into language that humans can understand based on the emotion intention. This process involves converting the animal's emotion and intention into a specific text or speech output, i.e., "the language conversion result", so that humans can intuitively understand the animal's "language" and achieve effective communication between humans and animals.

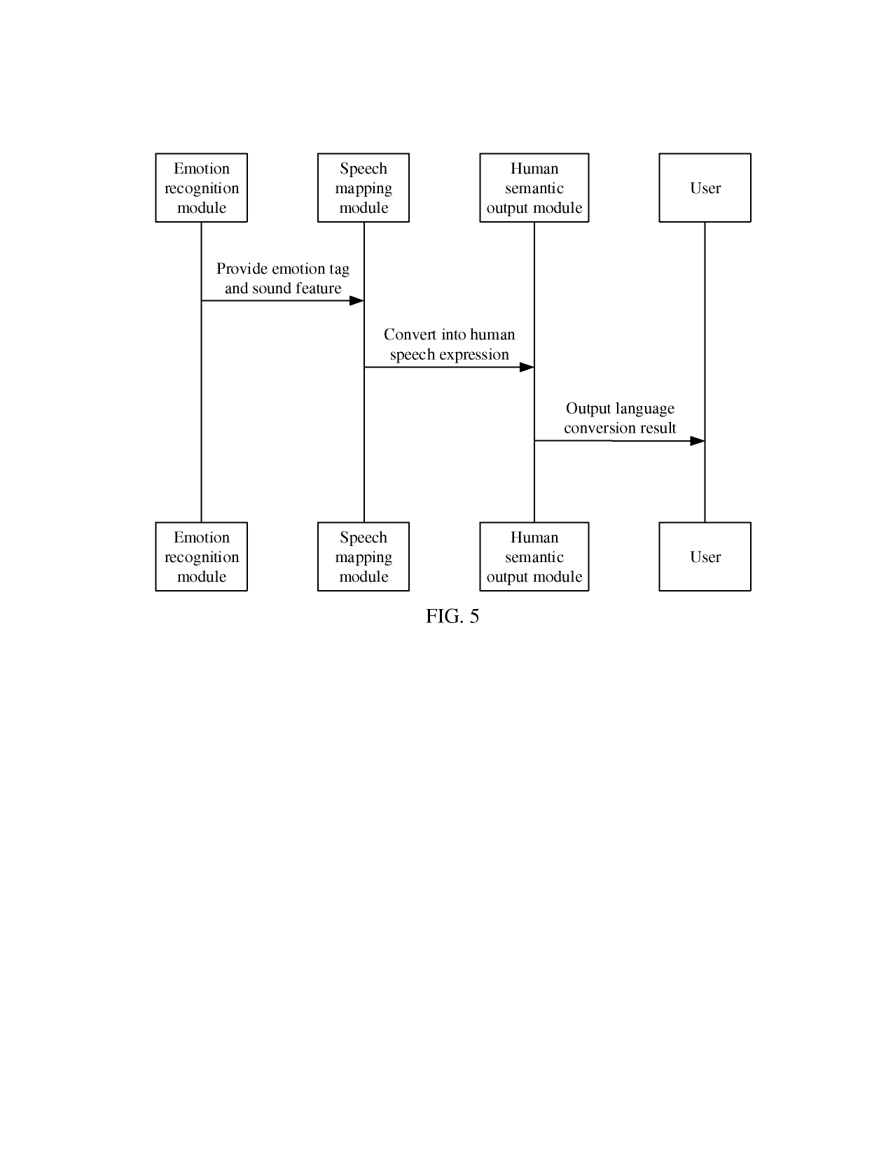

To facilitate understanding of the solution in the embodiments of the present disclosure, an example is given below, as shown in FIG. 5. FIG. 5 is a schematic diagram of a process of converting animal language into human language in an embodiment of the present disclosure. After obtaining the emotion recognition result, the emotion recognition module provides the animal's emotion tag and sound feature and then passes these information to the speech mapping module, which is responsible for converting the animal's sound feature into a speech expression that humans can understand. Next, the human semantic output module receives the converted speech information and outputs it as language conversion results, and finally presents these results to users to achieve real-time conversion from animal language to human language and emotional communication. The entire process involves emotion recognition, feature extraction and mapping, and human language generation, aiming to promote effective communication between humans and animals.

In this way, the emotion intention of the animal can be accurately captured and understood by using the pre-trained language model to perform semantic mapping of the emotion tag and sound vector, and then the language generator is used to convert the emotion intention into human language. This process not only enables the precise interpretation of the animal emotion, but also allows nonverbal communication from the animal to be converted into language that humans can understand, greatly promoting communication and understanding between humans and animals, and improving the transparency of animal emotional expression and the efficiency of communication.

In some optional embodiments, the method further includes: if specific sound data is detected and there is no historical record of emotion matching, labeling the specific sound data to obtain an updated emotion tag; and updating sample data dynamically according to the updated emotion tag, to adjust a model parameter according to the updated sample data.

Specifically, when specific animal sound data is detected and this sound data does not exist in the historical emotion matching records, that is, there is no previous emotion tag corresponding to this sound data, a labeling process will be triggered. Here, "labeling" refers to artificially assigning an emotion tag to the specific sound data. This tag describes the emotional state of the animal when making this sound, thus "obtaining an updated emotion tag". Subsequently, this newly labeled emotion tag is incorporated into the sample database, to implement the step of "updating sample data dynamically according to the updated emotion tag". The sample data updated in this way is used to adjust the model's parameter, that is, "adjust the model parameter according to the updated sample data", in order to optimize and improve the model's ability to recognize emotions for the newly emerging sound data, ensure that the system can adapt to new or uncommon animal sounds, and improve the accuracy of recognition and the adaptive learning ability of the system.

To facilitate understanding of the solution in the embodiments of the present disclosure, an example is given below. When the system encounters an unrecognizable vocal pattern or abnormal performance, the system will prompt the user to input a relevant tag or identification information. For example, the system detects a specific combination of action and sound without a clear historical record of matching emotional expression, and at this time, the user can add an annotation (such as "call for help" or "hungry" state) to this phonetic symbol through an interface. After the user performs manual annotation, the system will adjust the data parameters of the tag corresponding to the current audio and behavior, thereby updating the weights of the existing model. After the user completes annotation, the system updates the dataset at the right time, and continuously optimizes and retrains the relevant generative model, so that the system can gradually improve the recognition rate of similar or new categories of emotional events.

In this way, when specific sound data is detected and there is a lack of historical record of emotion matching, the manual annotation is performed and the emotion tag is updated accordingly, so that the sample database can be continuously expanded and enriched, thereby updating the model parameters dynamically, enhancing the system's ability to recognize emotions for new sound data, improving the model's adaptability and accuracy, and ensuring that the cross-species communication system can continuously evolve to better understand and respond to the animals' communication intentions. In some optional embodiments, the method further includes: collecting multimodal data within a preset time window to obtain emotional change data of the animal; performing feature extraction on the emotional change data to obtain an emotional change feature; and updating an emotion tag according to a difference between an emotional change feature of a current time window and an emotional change feature of a previous time window.

Specifically, the "preset time window" refers to a specific time period set for analyzing emotional changes of the animal. During this time period, "the multimodal data is collected", including information such as sounds, behaviors and physical signs of the animal, to gather data on the animal emotional state. Next, the key information capable of representing the animal emotional change, i.e., the "emotional change feature", is identified from these data through the "feature extraction" process. Then, the differences between the emotion features extracted in the current time window and the features in the previous time window are compared. If these differences indicate a significant change in the animal's emotional state, "the emotion tag is updated" according to this change to reflect the animal's latest emotional state. This process involves continuous monitoring and dynamic analysis of the animal's emotional state to ensure the real-time nature and accuracy of emotion recognition.

In this way, by collecting the multimodal data of the animal within the preset time window, the emotional change of the animal can be comprehensively captured and the feature extraction can be performed on these data to identify the emotional change feature. Secondly, by updating the emotion tag dynamically according to the difference between emotion features in the current and previous time windows, the real-time and accurate monitoring and response to the animal's emotional state can be achieved. This process not only improves the accuracy of emotion recognition, but also enhances the real-time nature and depth of communication between humans and animals, enabling humans to better understand and respond to the emotional requirements of animals. In some optional embodiments, the step of collecting the multimodal data within the preset time window to obtain the emotional change data of the animal includes: collecting the multimodal data within the preset time window; and inputting the multimodal data into an emotion period recognition model, to obtain the emotional change data of the animal through the emotion period recognition model.

Specifically, the "preset time window" refers to a specific time period set for monitoring and analyzing emotional changes of the animal. During this time period, the "multimodal data" is collected, including different types of information such as sounds, behaviors and physical signs of the animal. These data are then input into the "emotion period recognition model. This model is an algorithm specifically designed to process and analyze time series data, such as a Long Short-Term Memory Network (LSTM) or a Gated Recurrent Unit (GRU). The model analyzes the multimodal data to identify and extract features related to the animal's emotional state, thereby "obtaining the emotional change data of the animal". These data reflect the emotional state of the animal within the consecutive time window, providing a basis for further emotion recognition and tag update. In this way, the solution can achieve dynamic tracking and accurate identification of the animal emotional state.

Thus, the precise capture and analysis of the animal emotional change can be achieved by collecting the multimodal data of the animal within the preset time window and inputting the data into the emotion period recognition model. This method can recognize and understand the emotional dynamics of the animal over a continuous time period, thus providing the richer and more accurate emotional change data. These data not only enhance the depth and real-time nature of emotion recognition, but also improve the quality and efficiency of communication between humans and animals, enabling humans to respond to animals' emotional requirements and behavioral changes more meticulously and promptly.

In some optional embodiments, the step of updating the emotion tag according to the difference between the emotional change feature of the current time window and the emotional change feature of the previous time window includes: calculating an Euclidean distance difference between the emotional change feature of the current time window and the emotional change feature of the previous time window to obtain an emotion difference; and if the emotion difference exceeds a preset emotion difference threshold, upgrading the emotion tag to obtain an updated emotion tag.

Specifically, "the emotional change feature of the current time window" refers to an emotion-related feature extracted from the multimodal data of the animal within a specific time period, while "the emotional change feature of the previous time window" refers to a corresponding feature within the previous time period. A quantified "emotion difference" can be obtained by calculating the "Euclidean distance difference" between the emotion features of these two time windows, namely a method for measuring a distance between two points in a multi-dimensional space.

The Euclidean distance difference between the emotional change feature of the current time window and the emotional change feature of the previous time window satisfies a formula of: [D(W_i,W_{i-1})=\sqrt{\sum_{n=1}^{N}(x_{i,n}-x_{i-1,n})^2}], where D(W_i,W_{i-1}) represents the Euclidean distance between emotional change features of the i-th time window and the (i-1)-th time window, and this distance is used to quantify the degree of change in emotion features within two consecutive time windows. x_{i,n} represents the value of the n-th feature within the i-th time window. These features may include: pitch, rhythm and amplitude of sound, frequency of behavior, and change in heart rate of physical sign, etc. x_{i-1,n} represents the value of the n-th feature within the (i-1)-th time window. sum represents the summation operation, and sqrt represents the operation of calculating the square value.

The emotion difference is obtained by calculating the Euclidean distance difference between the emotional change feature of the current time window and the emotional change feature of the previous time window. This difference reflects the degree of change in the animal's emotional state within two consecutive time windows. If this difference exceeds the "preset emotion difference threshold", i.e., a preset boundary, indicating a significant change in the animal's emotional state, then the "emotion tag upgrade" will be triggered. Based on the emotion difference and change pattern, the emotion tag is updated from one state (such as "alert") to another tag (such as "anxiety") that is more in line with the current emotional state, thereby obtaining the "updated emotion tag". This process makes emotion recognition more dynamic and accurate, and can respond in real time to the significant change in animal emotion.

In this way, the degree of change in the animal's emotional state can be quantified by calculating the Euclidean distance difference between the emotional change features in the current and previous time windows, to obtain the emotion difference. When the emotion difference exceeds the preset threshold, the emotion tag will be automatically upgraded to obtain the updated emotion tag. This method makes emotion recognition more sensitive and accurate, and can capture and respond to significant changes in animal emotions in real time, thereby improving the quality and efficiency of communication between humans and animals, and ensuring that humans can understand and respond to the emotional requirements of animals in a timely and appropriate manner.

In some optional embodiments, the method further includes: scoring an emotion weight corresponding to each time window to obtain an emotion weight score; and accumulating emotion weight scores corresponding to similar emotion time windows within a continuous time period, and updating the emotion tag if an accumulated result is greater than a set upgrade threshold.

Specifically, the "emotion weight score" refers to the process of quantitatively assessing the emotional state of the animal within each time window, where each time window contains multimodal data collected within a specific time period. The emotion features are extracted by analyzing these data, and different weights are assigned according to the strength or significance of the features. Then, the emotion weight score for each time window is calculated. Next, the weight scores of the time windows with similar emotion features within the continuous time period are accumulated. If the accumulated score exceeds the "set upgrade threshold", i.e., a predefined value used to judge whether the emotional state is significant enough to trigger the tag update, then the emotion tag is updated to reflect the change in the animal's emotional state. This process makes emotion recognition more dynamic and accurate, and can respond in real time to continuous changes in animal emotion.

In this way, this solution can quantify and track the emotional change of the animal over a continuous time period by assigning a weight to and scoring the emotional state within each time window. The weight scores of the time windows with similar emotion features are accumulated. When the accumulated result exceeds the preset upgrade threshold, the emotion tag will be automatically updated to reflect the animal's current emotional state more accurately. This method improves the sensitivity and adaptability of emotion recognition, making communication between humans and animals more accurate and timely, and thus enhancing the ability to understand and respond to to animals' emotional requirements.

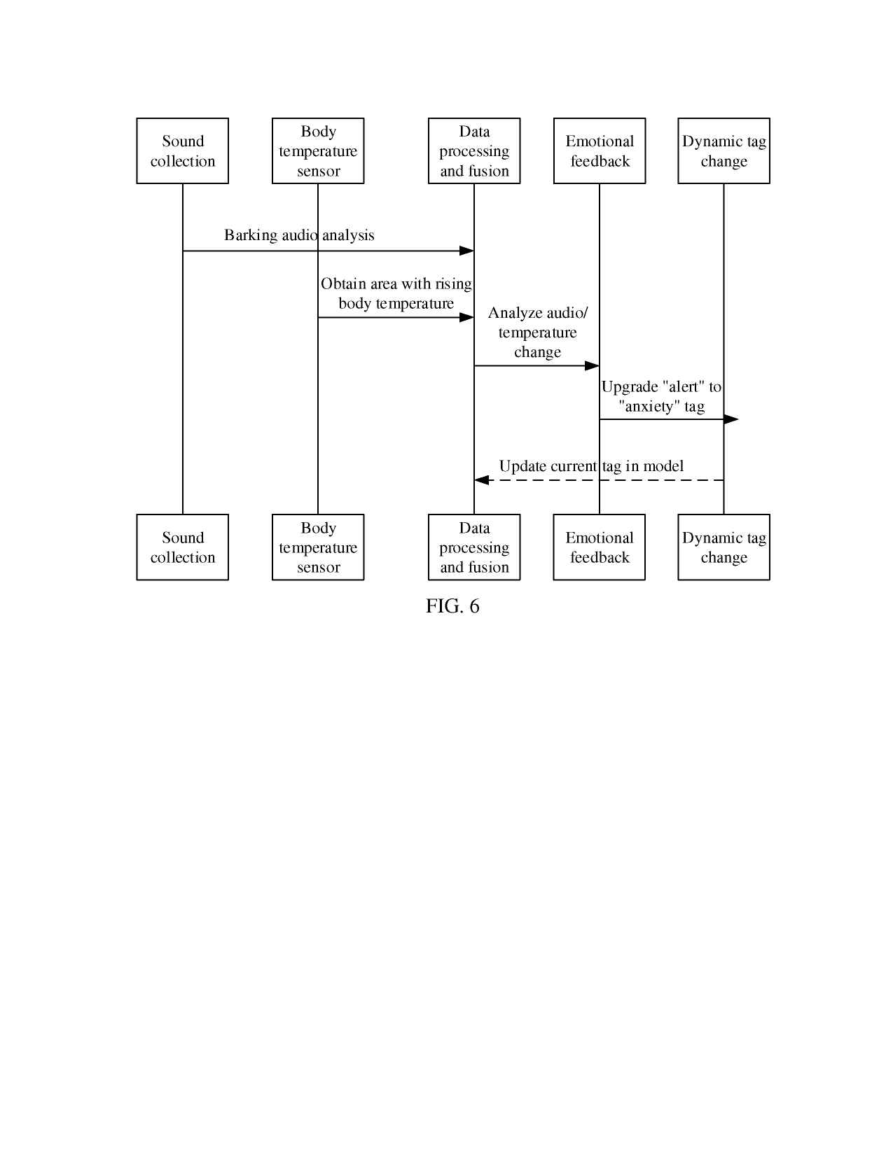

To facilitate understanding of the solution in the embodiments of the present disclosure, referring to FIG. 6, FIG. 6 is a schematic diagram of a process of updating the emotion tag in an embodiment of the present disclosure. The barking audio and body temperature data of the animal are collected by a sound collector and a body temperature sensor, and these data are then fed into a data processing and fusion module for analysis to identify audio features and changes in body temperature. The analysis result is used for emotional feedback. Based on the emotional meanings of the audio and changes in body temperature, the emotion tag of the animal is dynamically adjusted, for example, the "alert" state is upgraded to "anxiety". This process involves real-time monitoring and continuous assessment of the emotional state to ensure that the change in the animal's emotional state can be reflected in the tag update in a timely manner, thereby improving the accuracy in understanding and responding to the animal's emotional state.

In this way, the subtle changes in animal emotion can be monitored and quantified in real time by collecting the emotional change data of the animal within the continuous time window and extracting emotional change features from these data. By comparing the differences between these change features and standard emotion features and updating the emotion tag when the amount of change exceeds the preset threshold, the dynamic changes in animal emotion can be captured more accurately, thereby implementing continuous tracking and real-time response to the animal emotional state, improving the sensitivity and accuracy of emotion recognition, and enhancing the effectiveness of emotional communication between humans and animals.

The apparatus embodiments of the present application will be introduced below, and can be used to execute the method for converting animal language in the above embodiments of the present application. For details not disclosed in the apparatus embodiments of the present application, reference may be made to the above embodiments of the method for converting animal language in the present application.

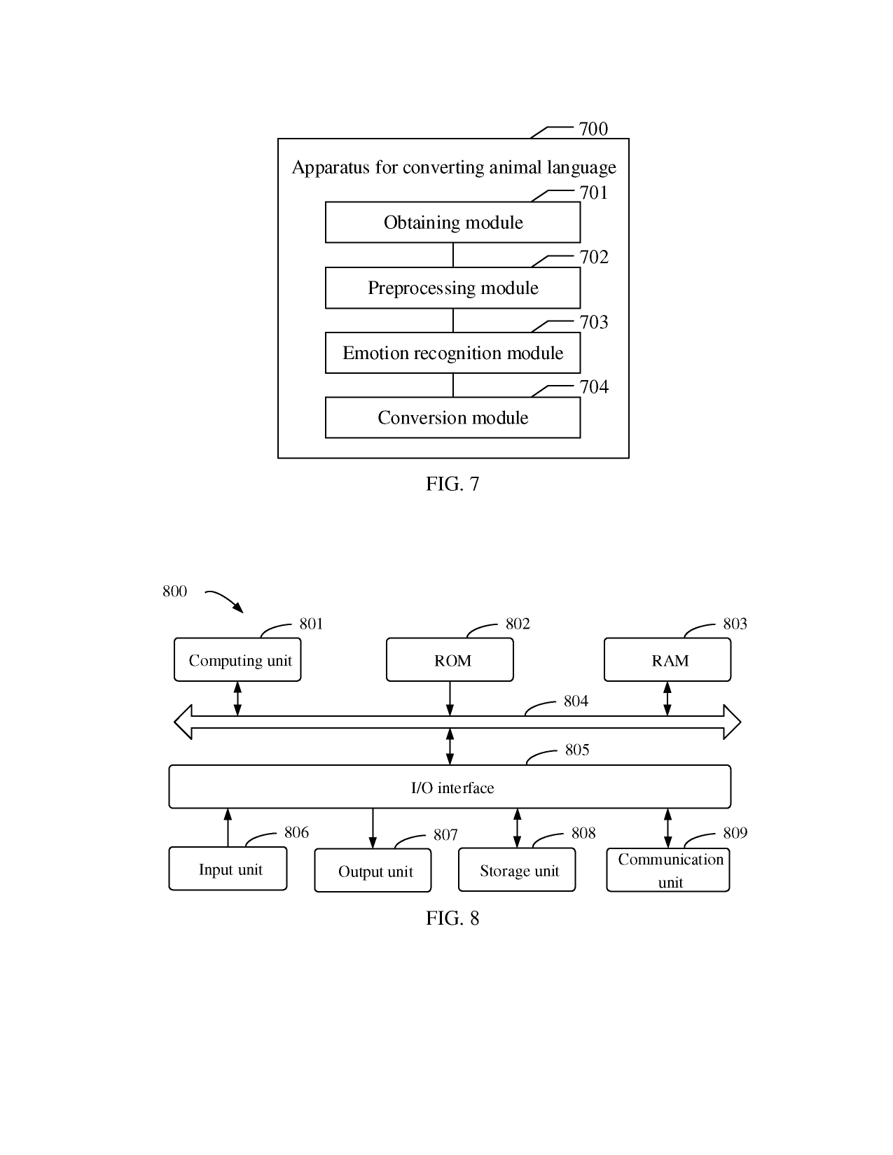

The present disclosure further provides an apparatus 700 for converting animal language, as shown in FIG. 7, including: an obtaining module 701 configured to obtain multimodal data related to an animal, where the multimodal data includes animal sound data, animal behavior data and animal physical sign data; a preprocessing module 702 configured to preprocess the multimodal data to obtain fused multimodal data; an emotion recognition module 703 configured to recognize current emotion of the animal according to the fused multimodal data to obtain an emotion recognition result of the animal; and a conversion module 704 configured to perform semantic mapping and language translation on the emotion recognition result to convert animal language into human language to obtain a language conversion result.

In some optional embodiments, the obtaining module 701 obtains the multimodal data related to the animal by: collecting sound wave information emitted by the animal to obtain the animal sound data; collecting body language and movement change of the animal to obtain the animal behavior data; and collecting physical and biological indicators of the animal to obtain the animal physical sign data.

In some optional embodiments, the preprocessing module 702 preprocesses the multimodal data to obtain the fused multimodal data by: denoising the multimodal data for data cleaning to obtain cleaned multimodal data; normalizing the cleaned multimodal data to obtain normalized multimodal data; and performing time series alignment and fusion on the normalized multimodal data to obtain the fused multimodal data.

In some optional embodiments, the emotion recognition module 703 recognizes the current emotion of the animal according to the fused multimodal data to obtain the emotion recognition result of the animal by: performing sound feature extraction, visual motion feature extraction and physical sign change analysis on the fused multimodal data by a deep learning model to obtain a multimodal feature vector; and performing emotion analysis on the multimodal feature vector by a generative adversarial network to obtain the emotion recognition result of the animal.

In some optional embodiments, the conversion module 704 performs semantic mapping and language translation on the emotion recognition result to convert animal language into human language to obtain the language conversion result by: extracting an emotion tag and a sound feature from the emotion recognition result, and converting the sound feature into a standardized sound vector; mapping the emotion tag and the sound vector semantically by a pre-trained language model to obtain an emotion intention; and performing language translation on the emotion intention by a language generator to generate corresponding human language, to obtain the language conversion result.

In some optional embodiments, the apparatus further includes a first update module configured to: if specific sound data is detected and there is no historical record of emotion matching, label the specific sound data to obtain an updated emotion tag; and update sample data dynamically according to the updated emotion tag, to adjust a model parameter according to the updated sample data.

In some optional embodiments, the apparatus further includes a second update module configured to: collect multimodal data within a preset time window to obtain emotional change data of the animal; perform feature extraction on the emotional change data to obtain an emotional change feature; and update an emotion tag according to a difference between an emotional change feature of a current time window and an emotional change feature of a previous time window.

In some optional embodiments, the second update module collects the multimodal data within the preset time window to obtain the emotional change data of the animal by: collecting the multimodal data within the preset time window; and inputting the multimodal data into an emotion period recognition model, to obtain the emotional change data of the animal through the emotion period recognition model.

In some optional embodiments, the second update module updates the emotion tag according to the difference between the emotional change feature of the current time window and the emotional change feature of the previous time window by: calculating an Euclidean distance difference between the emotional change feature of the current time window and the emotional change feature of the previous time window to obtain an emotion difference; and if the emotion difference exceeds a preset emotion difference threshold, upgrading the emotion tag to obtain an updated emotion tag.

In some optional embodiments, the second update module is configured to: score an emotion weight corresponding to each time window to obtain an emotion weight score; and accumulate emotion weight scores corresponding to similar emotion time windows within a continuous time period, and update the emotion tag if an accumulated result is greater than a set upgrade threshold.

In the technical solution of the present disclosure, the acquisition, storage and application of the user's personal information involved are in compliance with relevant laws and regulations, and do not violate public order and good customs.

According to the embodiments of the present disclosure, the present disclosure also provides an electronic device, a readable storage medium and a computer program product.

FIG. 8 shows a schematic block diagram of an exemplary electronic device 800 that may be used to implement the embodiments of the present disclosure. The electronic device is intended to represent various forms of digital computers, such as a laptop, a desktop, a workstation, a personal digital assistant, a server, a blade server, a mainframe computer, and other suitable computers. The electronic device may also represent various forms of mobile devices, such as a personal digital processing, a cellular phone, a smart phone, a wearable device and other similar computing devices. The components shown herein, their connections and relationships, and their functions are merely examples, and are not intended to limit the implementation of the present disclosure described and/or required herein.

As shown in FIG. 8, the electronic device 800 includes a computing unit 801 that may perform various appropriate actions and processes according to a computer program stored in a Read-Only Memory (ROM) 802 or a computer program loaded from a storage unit 808 into a Random Access Memory (RAM) 803. Various programs and data required for an operation of device 800 may also be stored in the RAM 803. The computing unit 801, the ROM 802 and the RAM 803 are connected to each other through a bus 804. The input/output (I/O) interface 805 is also connected to the bus 804.

A plurality of components in the device 800 are connected to the I/O interface 805, and include an input unit 806 such as a keyboard, a mouse, or the like; an output unit 807 such as various types of displays, speakers, or the like; the storage unit 808 such as a magnetic disk, an optical disk, or the like; and a communication unit 809 such as a network card, a modem, a wireless communication transceiver, or the like. The communication unit 809 allows the device 800 to exchange information/data with other devices through a computer network such as the Internet and/or various telecommunication networks.

The computing unit 801 may be various general-purpose and/or special-purpose processing components with processing and computing capabilities. Some examples of the computing unit 801 include, but are not limited to, a Central Processing Unit (CPU), a Graphics Processing Unit (GPU), various dedicated Artificial Intelligence (AI) computing chips, various computing units that run machine learning model algorithms, a Digital Signal Processor (DSP), and any appropriate processors, controllers, microcontrollers, or the like. The computing unit 801 performs various methods and processes described above, such as the method for converting animal language. For example, in some implementations, the method for converting animal language may be implemented as a computer software program tangibly contained in a computer-readable medium, such as the storage unit 808. In some implementations, a part or all of the computer program may be loaded and/or installed on the device 800 via the ROM 802 and/or the communication unit 809. When the computer program is loaded into the RAM 803 and executed by the computing unit 801, one or more steps of the method for converting animal language described above may be performed. Alternatively, in other implementations, the computing unit 801 may be configured to perform the method for converting animal language by any other suitable means (e.g., by means of firmware).

Various implementations of the system and technologies described above herein may be implemented in a digital electronic circuit system, an integrated circuit system, a Field Programmable Gate Array (FPGA), an Application Specific Integrated Circuit (ASIC), an Application Specific Standard Product (ASSP), a System on Chip (SOC), a Complex Programmable Logic Device (CPLD), a computer hardware, firmware, software, and/or a combination thereof. These various implementations may be implemented in one or more computer programs, and the one or more computer programs may be executed and/or interpreted on a programmable system including at least one programmable processor. The programmable processor may be a special-purpose or general-purpose programmable processor, may receive data and instructions from a storage system, at least one input device, and at least one output device, and transmit the data and the instructions to the storage system, the at least one input device, and the at least one output device.

The program code for implementing the method of the present disclosure may be written in any combination of one or more programming languages. The program code may be provided to a processor or controller of a general-purpose computer, a special-purpose computer or other programmable data processing devices, which enables the program code, when executed by the processor or controller, to cause the function/operation specified in the flowchart and/or block diagram to be implemented. The program code may be completely executed on a machine, partially executed on the machine, partially executed on the machine as a separate software package and partially executed on a remote machine, or completely executed on the remote machine or a server.

In the context of the present disclosure, a machine-readable medium may be a tangible medium, which may contain or store a procedure for use by or in connection with an instruction execution system, device or apparatus. The machine-readable medium may be a machine-readable signal medium or a machine-readable storage medium. The machine-readable medium may include, but is not limited to, an electronic, magnetic, optical, electromagnetic, infrared or semiconductor system, device or apparatus, or any suitable combination thereof. More specific examples of the machine-readable storage medium may include electrical connections based on one or more lines, a portable computer disk, a hard disk, a Random Access Memory (RAM), a Read-Only Memory (ROM), an Erasable Programmable Read-Only Memory (EPROM or a flash memory), an optical fiber, a portable Compact Disc Read-Only Memory (CD-ROM), an optical storage device, a magnetic storage device, or any suitable combination thereof.

In order to provide interaction with a user, the system and technologies described herein may be implemented on a computer that has: a display apparatus (e.g., a cathode ray tube (CRT) or a Liquid Crystal Display (LCD) monitor) for displaying information to the user; and a keyboard and a pointing device (e.g., a mouse or a trackball) through which the user may provide input to the computer. Other types of devices may also be used to provide interaction with the user. For example, feedback provided to the user may be any form of sensory feedback (e.g., visual feedback, auditory feedback, or tactile feedback), and the input from the user may be received in any form (including an acoustic input, a voice input, or a tactile input).

The system and technologies described herein may be implemented in a computing system (which serves as, for example, a data server) including a back-end component, or in a computing system (which serves as, for example, an application server) including a middleware, or in a computing system including a front-end component (e.g., a user computer with a graphical user interface or web browser through which the user may interact with the implementation of the system and technologies described herein), or in a computing system including any combination of the back-end component, the middleware component, or the front-end component. The components of the system may be connected to each other through any form or kind of digital data communication (e.g., a communication network). Examples of the communication network include a Local Area Network (LAN), a Wide Area Network (WAN), and the Internet.

A computer system may include a client and a server. The client and server are generally far away from each other and usually interact with each other through a communication network. A relationship between the client and the server is generated by computer programs running on corresponding computers and having a client-server relationship with each other. The server may be a cloud server, a distributed system server, or a blockchain server.

It should be understood that, the steps may be reordered, added or removed by using the various forms of the flows described above. For example, the steps recorded in the present disclosure can be performed in parallel, in sequence, or in different orders, as long as a desired result of the technical scheme disclosed in the present disclosure can be realized, which is not limited herein.

The foregoing specific implementations do not constitute a limitation on the protection scope of the present disclosure. Those having ordinary skill in the art should understand that, various modifications, combinations, sub-combinations and substitutions may be made according to a design requirement and other factors. Any modification, equivalent replacement, improvement or the like made within the spirit and principle of the present disclosure shall be included in the protection scope of the present disclosure.

What is claimed is:

1. A method for converting animal language, comprising:
obtaining multimodal data related to an animal, wherein the multimodal data comprises animal sound data, animal behavior data and animal physical sign data;
preprocessing the multimodal data to obtain fused multimodal data;
recognizing current emotion of the animal according to the fused multimodal data to obtain an emotion recognition result of the animal; and
performing semantic mapping and language translation on the emotion recognition result to convert animal language into human language to obtain a language conversion result.

2. The method of claim 1, wherein the obtaining of the multimodal data related to the animal, comprises:
collecting sound wave information emitted by the animal to obtain the animal sound data;
collecting body language and movement change of the animal to obtain the animal behavior data; and
collecting physical and biological indicators of the animal to obtain the animal physical sign data.

3. The method of claim 1, wherein the preprocessing of the multimodal data to obtain the fused multimodal data, comprises:
denoising the multimodal data for data cleaning to obtain cleaned multimodal data;
normalizing the cleaned multimodal data to obtain normalized multimodal data; and
performing time series alignment and fusion on the normalized multimodal data to obtain the fused multimodal data.

4. The method of claim 1, wherein the recognizing of the current emotion of the animal according to the fused multimodal data to obtain the emotion recognition result of the animal, comprises:
performing sound feature extraction, visual motion feature extraction and physical sign change analysis on the fused multimodal data by a deep learning model to obtain a multimodal feature vector; and
performing emotion analysis on the multimodal feature vector by a generative adversarial network to obtain the emotion recognition result of the animal.

5. The method of claim 1, wherein the performing of the semantic mapping and language translation on the emotion recognition result to convert the animal language into the human language to obtain the language conversion result, comprises:
extracting an emotion tag and a sound feature from the emotion recognition result, and converting the sound feature into a standardized sound vector;
mapping the emotion tag and the sound vector semantically by a pre-trained language model to obtain an emotion intention; and
performing language translation on the emotion intention by a language generator to generate corresponding human language, to obtain the language conversion result.

6. The method of claim 1, further comprising:
in a case of specific sound data is detected and there is no historical record of emotion matching, labeling the specific sound data to obtain an updated emotion tag; and
updating sample data dynamically according to the updated emotion tag, to adjust a model parameter according to the updated sample data.

7. The method of claim 1, further comprising:
collecting multimodal data within a preset time window to obtain emotional change data of the animal;
performing feature extraction on the emotional change data to obtain an emotional change feature; and
updating an emotion tag according to a difference between an emotional change feature of a current time window and an emotional change feature of a previous time window.

8. The method of claim 7, wherein the collecting of the multimodal data within the preset time window to obtain the emotional change data of the animal, comprises:
collecting the multimodal data within the preset time window; and
inputting the multimodal data into an emotion period recognition model, to obtain the emotional change data of the animal through the emotion period recognition model.

9. The method of claim 7, wherein the updating of the emotion tag according to the difference between the emotional change feature of the current time window and the emotional change feature of the previous time window, comprises:
calculating an Euclidean distance difference between the emotional change feature of the current time window and the emotional change feature of the previous time window to obtain an emotion difference; and
in a case of the emotion difference exceeds a preset emotion difference threshold, upgrading the emotion tag to obtain an updated emotion tag.

10. The method of claim 9, further comprising:
scoring an emotion weight corresponding to each time window to obtain an emotion weight score; and
accumulating emotion weight scores corresponding to similar emotion time windows within a continuous time period, and updating the emotion tag, in a case of an accumulated result is greater than a set upgrade threshold.

11. An electronic device, comprising:
at least one processor; and
a memory connected in communication with the at least one processor;
wherein the memory stores an instruction executable by the at least one processor, and the instruction, when executed by the at least one processor, enables the at least one processor to execute:
obtaining multimodal data related to an animal, wherein the multimodal data comprises animal sound data, animal behavior data and animal physical sign data;
preprocessing the multimodal data to obtain fused multimodal data;
recognizing current emotion of the animal according to the fused multimodal data to obtain an emotion recognition result of the animal; and
performing semantic mapping and language translation on the emotion recognition result to convert animal language into human language to obtain a language conversion result.

12. The electronic device of claim 11, wherein the instruction, when executed by the at least one processor, enables the at least one processor to execute the obtaining of the multimodal data related to the animal, by:
collecting sound wave information emitted by the animal to obtain the animal sound data;
collecting body language and movement change of the animal to obtain the animal behavior data; and
collecting physical and biological indicators of the animal to obtain the animal physical sign data.

13. The electronic device of claim 11, wherein the instruction, when executed by the at least one processor, enables the at least one processor to execute the preprocessing of the multimodal data to obtain the fused multimodal data, by:
denoising the multimodal data for data cleaning to obtain cleaned multimodal data;
normalizing the cleaned multimodal data to obtain normalized multimodal data; and
performing time series alignment and fusion on the normalized multimodal data to obtain the fused multimodal data.

14. The electronic device of claim 11, wherein the instruction, when executed by the at least one processor, enables the at least one processor to execute the recognizing of the current emotion of the animal according to the fused multimodal data to obtain the emotion recognition result of the animal, by:
performing sound feature extraction, visual motion feature extraction and physical sign change analysis on the fused multimodal data by a deep learning model to obtain a multimodal feature vector; and
performing emotion analysis on the multimodal feature vector by a generative adversarial network to obtain the emotion recognition result of the animal.

15. The electronic device of claim 11, wherein the instruction, when executed by the at least one processor, enables the at least one processor to execute the performing of the semantic mapping and language translation on the emotion recognition result to convert the animal language into the human language to obtain the language conversion result, by:
extracting an emotion tag and a sound feature from the emotion recognition result, and converting the sound feature into a standardized sound vector;
mapping the emotion tag and the sound vector semantically by a pre-trained language model to obtain an emotion intention; and
performing language translation on the emotion intention by a language generator to generate corresponding human language, to obtain the language conversion result.

16. A non-transitory computer-readable storage medium storing a computer instruction thereon, wherein the computer instruction is used to cause a computer to execute:
obtaining multimodal data related to an animal, wherein the multimodal data comprises animal sound data, animal behavior data and animal physical sign data;
preprocessing the multimodal data to obtain fused multimodal data;
recognizing current emotion of the animal according to the fused multimodal data to obtain an emotion recognition result of the animal; and
performing semantic mapping and language translation on the emotion recognition result to convert animal language into human language to obtain a language conversion result.

17. The non-transitory computer-readable storage medium of claim 16, wherein the computer instruction is used to cause the computer to execute the obtaining of the multimodal data related to the animal, by:
collecting sound wave information emitted by the animal to obtain the animal sound data;
collecting body language and movement change of the animal to obtain the animal behavior data; and
collecting physical and biological indicators of the animal to obtain the animal physical sign data.

18. The non-transitory computer-readable storage medium of claim 16, wherein the computer instruction is used to cause the computer to execute the preprocessing of the multimodal data to obtain the fused multimodal data, by:
denoising the multimodal data for data cleaning to obtain cleaned multimodal data;
normalizing the cleaned multimodal data to obtain normalized multimodal data; and
performing time series alignment and fusion on the normalized multimodal data to obtain the fused multimodal data.

19. The non-transitory computer-readable storage medium of claim 16, wherein the computer instruction is used to cause the computer to execute the recognizing of the current emotion of the animal according to the fused multimodal data to obtain the emotion recognition result of the animal, by:
performing sound feature extraction, visual motion feature extraction and physical sign change analysis on the fused multimodal data by a deep learning model to obtain a multimodal feature vector; and
performing emotion analysis on the multimodal feature vector by a generative adversarial network to obtain the emotion recognition result of the animal.

20. The non-transitory computer-readable storage medium of claim 16, wherein the computer instruction is used to cause the computer to execute the performing of the semantic mapping and language translation on the emotion recognition result to convert the animal language into the human language to obtain the language conversion result, by:
extracting an emotion tag and a sound feature from the emotion recognition result, and converting the sound feature into a standardized sound vector;
mapping the emotion tag and the sound vector semantically by a pre-trained language model to obtain an emotion intention; and
performing language translation on the emotion intention by a language generator to generate corresponding human language, to obtain the language conversion result.
